(19) United States
(12) Patent Application Publication
(10) Pub. No.: US 2022/0142526 A1
(43) Pub. Date: May 12, 2022

(54) SYSTEM AND METHOD FOR AUTOMATIC DETECTION OF BLOOD LACTATE LEVELS

(71) Applicants: Jacob Levman, Clydesdale (CA); Koichi Sughimoto, Chiba-city (JP)

(72) Inventors: Jacob Levman, Clydesdale (CA); Koichi Sughimoto, Chiba-city (JP)

(21) Appl. No.: 17/525,417

(22) Filed: Nov. 12, 2021

Related U.S. Application Data
(60) Provisional application No. 63/112,815, filed on Nov. 12, 2020.

(51) Int. Cl. A61B 5/145; A61B 5/00; A61B 5/021; A61B 5/0205; A61B 5/318; G16H 50/30; G16H 10/60; G06N 20/00

(52) U.S. Cl. CPC A61B 5/14546; A61B 5/7264; A61B 5/7275; A61B 5/021; A61B 5/0205; A61B 2503/06; G16H 50/30; G16H 10/60; G06N 20/00; A61B 2503/04; A61B 5/318

(57) ABSTRACT

Methods and systems are provided herein for automatically determining blood lactate levels. The method can comprise receiving bio-signal data associated with a patient; processing the bio-signal data to extract one or more bio-signal data features; generating a tensor dataset comprising the one or more bio-signal data features; and processing the tensor dataset using a machine learning model to generate an estimated blood lactate level.

### CROSS-REFERENCE TO RELATED APPLICATIONS

This application claims the benefit of U.S. Provisional Application No. 63/112,815 filed on Nov. 12, 2020, which is incorporated by reference herein in its entirety.

### FIELD

The described embodiments relate to detection of blood lactate levels, and in particular, to a system and method for automatic detection of blood lactate levels.

### INTRODUCTION

The following is not an admission that anything discussed below is part of the prior art or part of the common general knowledge of a person skilled in the art.

Measurements of patient blood lactate levels can provide valuable indications of various medical conditions including, inter alia, congestive heart failure, sepsis, lung disease, and liver and/or kidney disease. In many cases, pediatric intensive care units (PICUs) may monitor blood lactate levels for newborns or premature babies—who are otherwise in critical condition—for early detection of possible heart failure, cardiac arrest, renal failure, etc.

Conventional techniques for blood lactate level monitoring often rely on analyzing patient blood draw samples, whereby each new lactate level measurement can require the patient to submit a new blood sample. However, the requirement for new patient blood draw samples for each new lactate level measurement may be highly invasive and may present particular challenges for neonatal blood lactate level detection. In many cases, excessive blood draw samples from a newborn or premature baby can serve to increase the risk of causing anemia rather than decrease the baby's risk of cardiac arrest.

In view of the foregoing, there is a desire for a less invasive process for monitoring patient blood lactate levels, and with practical applications for neonatal blood lactate level detection.

### SUMMARY OF THE VARIOUS EMBODIMENTS

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In a broad aspect, there is provided a method of determining blood lactate levels, the method comprising: receiving bio-signal data associated with a patient; processing the bio-signal data to extract one or more bio-signal data features; generating a tensor dataset comprising the one or more bio-signal data features; and processing the tensor dataset using a machine learning model to generate an estimated blood lactate level.

In some embodiments, the method further comprises based on the estimated blood locate level, generating a prediction of risk of cardiac arrest for the patient.

In some embodiments, the patient is a pediatric patient.

In some embodiments, the method further comprises updating the tensor dataset to comprise case-specific data, prior to processing the tensor dataset.

In some embodiments, the case-specific data comprises previous blood draw data for the patient.

In some embodiments, the case-specific data comprises at least one of biographical data and observational data for the patient.

In some embodiments, the machine learning model is a convolutional neural network.

In some embodiments, the machine learning model is based on support vector machines.

In some embodiments, the machine learning model is generated using a random forest algorithm.

In some embodiments, the processing of the tensor dataset comprises using cross-validation.

In some embodiments, the cross-validation comprises one of K-fold and Efron's bootstrap.

In some embodiments, the cross-validation is K-fold cross-validation, and K is in a range of 1 to 20.

In some embodiments, the machine learning model is determined using adaptive boosting.

In some embodiments, the one or more bio-signal data features comprise second bio-signal data features, and the method further comprises: processing the bio-signal data to generate one or more first bio-signal data features; determining if the one or more first bio-signal data features are greater than a predetermined threshold; and in response to the determination, processing the tensor dataset using the machine learning model.

In some embodiments, the method further comprises applying a treatment to the pediatric patient in response to the estimated blood lactate level.

In some embodiments, the bio-signal data comprises at least one arterial blood pressure data or electrocardiogram (ECG) data.

In another broad aspect, there is provided a non-transitory computer readable medium storing computer program instructions which, when executed by at least one processor, cause the at least one processor to carry out the method comprising: receiving bio-signal data associated with a patient; processing the bio-signal data to extract one or more bio-signal data features; generating a tensor dataset comprising the one or more bio-signal data features; and processing the tensor dataset using a machine learning model to generate an estimated blood lactate level.

In still another broad aspect, there is provided a system for determining blood lactate levels, the system comprising: at least one processor; and a memory coupled to the at least one processor; the at least one processor being configured to carry out the method comprising: receiving bio-signal data associated with a patient; processing the bio-signal data to extract one or more bio-signal data features; generating a tensor dataset comprising the one or more bio-signal data features; and processing the tensor dataset using a machine learning model to generate an estimated blood lactate level.

### BRIEF DESCRIPTION OF THE DRAWINGS

A preferred embodiment of the present invention will now be described in detail with reference to the drawings, in which:

FIG. 1 is a system for automatic detection of patient blood lactate levels, in accordance with at least some embodiments;

FIG. 2 is a simplified hardware block diagram for an example computing device used for detecting patient blood lactate levels, according to some embodiments;

FIG. 3A is a process flow for an example method for automatic detection of patient blood lactate levels, according to some embodiments;

FIG. 3B is another process flow for an example method for automatic detection of patient blood lactate levels, according to some other embodiments;

FIG. 3C is still another process flow for an example method for automatic detection of patient blood lactate levels, according to still some other embodiments;

FIG. 4 are various illustrations for an example process for calculating an area under a cardiac waveform curve;

FIG. 5 are various plots illustrating example cardiac waveforms having different waveform peak sharpness;

FIG. 6A is a process flow for an example method of updating blood lactate level predictions based on new patient bio-signal data, in accordance with some embodiments;

FIG. 6B is a process flow for an example method of updating blood lactate level predictions based on updated patient blood draw data, in accordance with some embodiments;

FIG. 7 is a process flow for an example method for predicting the risk of cardiac arrest for a patient based on detected changes in blood lactate levels, in accordance with some embodiments; and

FIG. 8 is a process flow for an example method for training a machine learning model to automatically detect patient blood lactate levels.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity.

### DESCRIPTION OF VARIOUS EMBODIMENTS

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details, or with other methods, components, materials, etc. In other instances, well-known methods, procedures and components have not been described in detail since these are known to those skilled in the art. Furthermore, it should be noted that this description is not intended to limit the scope of the embodiments described herein, but rather as merely describing one or more exemplary implementations.

Unless the context requires otherwise, throughout the specification and claims which follow, the word “comprise” and variations thereof, such as, “comprises” and “comprising” are to be construed in an open, inclusive sense, that is as “including, but not limited to.”

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

Reference throughout this specification to “one embodiment” or “an embodiment” means that a particular feature, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

The headings and Abstract of the Disclosure provided herein are for convenience only and do not interpret the scope or meaning of the embodiments.

The terms “coupled” or “coupling” as used herein can have several different meanings depending in the context in which these terms are used. For example, the terms coupled or coupling may be used to indicate that an element or device can electrically, optically, or wirelessly send data to another element or device as well as receive data from another element or device.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Exemplary communicative pathways include, but are not limited to, electrically conductive pathways (e.g., electrically conductive wires, electrically conductive traces), magnetic pathways (e.g., magnetic media), optical pathways (e.g., optical fiber), electromagnetically radiative pathways (e.g., radio waves), or any combination thereof. Exemplary communicative couplings include, but are not limited to, electrical couplings, magnetic couplings, optical couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

The example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a keyboard, mouse, touchscreen, or the like), and at least one output device (e.g. a display screen, a printer, a wireless radio, or the like) depending on the nature of the device.

It should also be noted that there may be some elements that are used to implement at least part of one of the embodiments described herein that may be implemented via software that is written in a high-level computer programming language such as one that employs an object-oriented paradigm. Accordingly, the program code may be written in Java, C++, python or any other suitable programming language and may comprise modules or classes, as is known to those skilled in object-oriented programming. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, EEPROM, magnetic disk, optical disc) or a device that is readable by a general or special purpose programmable device. The software program code, when read by the programmable device, configures the programmable device to operate in a new, specific and predefined manner in order to perform at least one of the methods described herein.

The term “software application” or “application” refers to computer-executable instructions, particularly computer-executable instructions stored in a non-transitory medium, such as a non-volatile memory, and executed by a computer processor. The computer processor, when executing the instructions, may receive inputs and transmit outputs to any of a variety of input or output devices to which it is coupled. Software applications may include mobile applications or “apps” for use on mobile devices such as smartphones and tablets or other “smart” devices.

A software application can be, for example, a monolithic software application, built in-house by the organization and possibly running on custom hardware; a set of interconnected modular subsystems running on similar or diverse hardware; a software-as-a-service application operated remotely by a third party; third party software running on outsourced infrastructure, etc. In some cases, a software application also may be less formal, or constructed in ad hoc fashion, such as a programmable spreadsheet document that has been modified to perform computations for the organization's needs.

Software applications may be deployed to and installed on a computing device on which it is to operate. Depending on the nature of the operating system and/or platform of the computing device, an application may be deployed directly to the computing device, and/or the application may be downloaded from an application marketplace. For example, user of the user device may download the application through an app store such as the Apple App Store™ or Google™ Play™.

The description sets forth various embodiments of the systems, devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs executed by one or more computers (e.g., as one or more programs running on one or more computer systems), as one or more programs executed by on one or more controllers (e.g., microcontrollers) as one or more programs executed by one or more processors (e.g., microprocessors, central processing units, graphical processing units), as firmware, or as virtually any combination thereof, and that designing the circuitry and/or writing the code for the software and or firmware would be well within the skill of one of ordinary skill in the art in light of the teachings of this disclosure.

When logic is implemented as software and stored in memory, logic or information can be stored on any processor-readable medium for use by or in connection with any processor-related system or method. In the context of this disclosure, a memory is a processor-readable medium that is an electronic, magnetic, optical, or other physical device or means that contains or stores a computer and/or processor program. Logic and/or the information can be embodied in any processor-readable medium for use by or in connection with an instruction execution system, apparatus, or device, such as a computer-based system, processor-containing system, or other system that can fetch the instructions from the instruction execution system, apparatus, or device and execute the instructions associated with logic and/or information.

In the context of this specification, a “non-transitory computer-readable medium” can be any element that can store the program associated with logic and/or information for use by or in connection with the instruction execution system, apparatus, and/or device. The processor-readable medium can be, for example, but is not limited to, an electronic, magnetic, optical, electromagnetic, infrared, or semiconductor system, apparatus or device. More specific examples (a non-exhaustive list) of the computer readable medium would include the following: a portable computer diskette (magnetic, compact flash card, secure digital, or the like), a random access memory (RAM), a read-only memory (ROM), an erasable programmable read-only memory (EPROM, EEPROM, or Flash memory), a portable compact disc read-only memory (CDROM), digital tape, and other non-transitory media.

As noted elsewhere herein, conventional techniques for monitoring blood lactate levels—i.e., based on acquiring patient blood draw samples—can be highly invasive, and may not be ideally suited for continuous neonatal blood lactate level monitoring. In view of the foregoing, embodiments herein may generally provide for less invasive techniques for detection and on-going monitoring of patient blood lactate levels.

In particular, methods and systems herein provide for the use of a machine learning (ML) system that uses a trained model to estimate blood lactate levels for patients. In at least some embodiments, the ML system generates estimates of patient blood lactate levels based on monitored patient data, such as bio-signal data, as well as other patient data, collectively referred to herein as case-specific data. In at least some cases, the ML system generates estimated blood lactate levels in real-time, or near real-time, based on the monitored bio-signal data.

As the trained models described herein do not rely on new blood draw samples for each new determination of blood lactate levels, the provided methods and systems may permit for less invasive monitoring of lactate levels as compared to conventional techniques, and thus may have practical applications for neonatal blood lactate level detection.

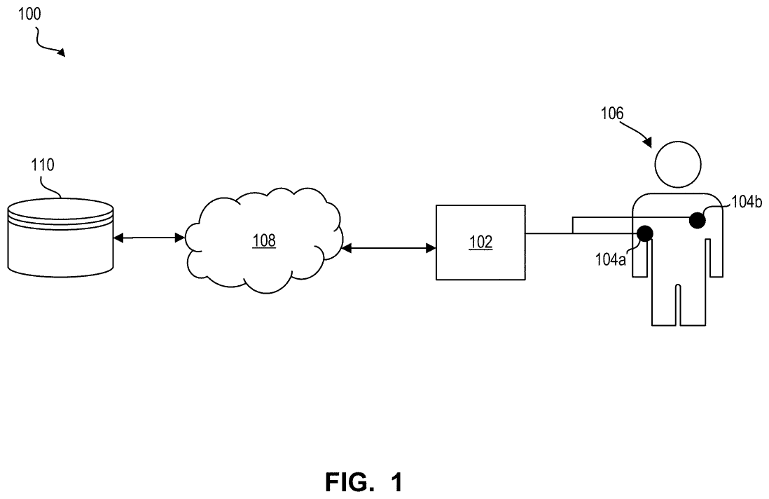

Referring now to FIG. 1, there is shown an example embodiment of a system 100 for automatic detection of patient blood lactate levels according to some embodiments. The system 100 provides the environment in which the methods described herein generally operate.

As shown, the system 100 may generally include a computing device 102 coupled, e.g., via network 108, to a data storage 110. In some cases, the computing device 102 may also communicate with one or more sensors and/or monitoring devices 104a, 104b applied to a patient 106.

Computing device 102 may be any suitable computing device capable of executing one or more software programs. For example, in various embodiments, the computing device 102 may be a mobile device such as a smartphone, tablet or laptop, as well as other computing devices such as wearable computing devices such as smartwatch. Computing device 102 may also be a non-mobile computer device, such as a desktop computer.

In embodiments described herein, computing device 102 may implement a machine learning model that, when trained, can predict patient blood lactate levels based on received bio-signal data and other case-specific data.

As shown, in some embodiments, computing device 102 may be coupled to one or more sensors and/or monitoring devices 104a, 104b, which can be used for detecting the bio-signal data of a patient for monitoring by computing device 102.

Sensors and/or monitoring devices 104 may include, for example, electrocardiograph (ECG) electrodes positioned over a patient's body 106 for acquiring ECG waveform data (e.g., a so-called ‘PQRST’ waveform). The sensors and/or monitoring devices 104 can also include a sphygmomanometer (e.g., a blood pressure cuff), or any other device suitable for measuring patient arterial blood pressure. In still other cases, sensors 104 may include one or more pulse oximeters, or similar devices for acquiring patient photoplethysmogram (PPG) data.

In various cases, bio-signal data—captured by one or more of the sensors and/or monitoring devices 104—may be relayed, or transmitted, directly to the computing device 102 (e.g., in real-time, or near real-time). In other embodiments, the sensors and/or monitoring devices 104 may not be coupled directly to the computing device 102, but rather, may couple to a bio-signal acquisition device (not shown). The bio-signal acquisition device may, in turn, directly couple to the computing device 102, or otherwise couple to the computing device 102 via network 108. In some cases, the bio-signal data may be stored following acquisition, e.g., for later analysis.

Data storage 110 can include one or more computer servers, each of which can include a storage memory, a processor and a network interface. The storage memory can comprise a random access memory (RAM), a read-only memory (ROM), an erasable programmable read-only memory (EPROM, EEPROM or Flash memory), one or more hard drives or other suitable data storage elements. The data storage 110 can also be a cloud storage and can include a number of servers, or computer nodes connected via network 108.

The data storage 110 stores data in relation to various patients (e.g., patient 106) and, in particular, can store case-specific data and bio-signal data for a patient. For example, data storage 110 may store bio-signal data (including historical bio-signal data), as well as case-specific data that includes blood draw data (e.g., prior blood lactate levels), biographical data (e.g., age, weight, height, disease status, etc.) and observational data (e.g., medical practitioner diagnoses or other inputs). In some cases, computing device 102 may communicate with the data storage 110 to request and/or receive relevant case-specific data and/or bio-signal data for use in predicting patient blood lactate levels. Computing device 102 may also transmit to data storage 110 updated blood draw data, such as blood lactate level determinations, for storage and/or subsequent retrieval.

Network 108 may be connected to the Internet. In various cases, the connection between the network and the Internet may be made, for example, via a firewall server. In some cases, there may be multiple links or firewalls, or both, between the communication network and the Internet. Some organizations may operate multiple networks or virtual networks, which can be Internetworked or isolated. The network 108 may be constructed from one or more computer network technologies, such as IEEE 802.3 (Ethernet), IEEE 802.11 and similar technologies. In some embodiments, network 108 may be omitted and the data storage may be directly coupled to computing device 102.

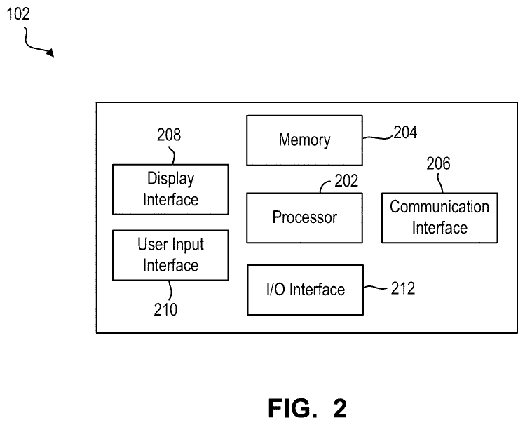

Referring now to FIG. 2, there is shown a simplified hardware block diagram for an example computing device 102 which can be used for automatic detection of patient blood lactate levels, in accordance with some embodiments.

As shown, the computing device 102 generally includes a processor 202 coupled, via a data bus, to one or more of a memory 204, a communication interface 206, a display interface 208, a user input interface 210 and an input/output (I/O) interface 212.

Processor 202 is a computer processor, such as a general purpose microprocessor. In some other cases, processor 202 may be a field programmable gate array (FPGA), an application specific integrated circuit (ASIC), a microcontroller, or any other suitable computer processor.

Processor 202 is coupled, via a computer data bus, to memory 204. Memory 204 may include both volatile and non-volatile memory. Non-volatile memory stores computer programs consisting of computer-executable instructions, which may be loaded into volatile memory for execution by processor 202 as needed. It will be understood by those of skill in the relevant art that references herein to the computing device 102 as carrying out a function, or acting in a particular way, can be understood as processor 202 executing instructions (e.g., a software program) stored in memory 204 and transmitting or receiving inputs and outputs via one or more interfaces where appropriate. Memory 204 may also store data input to, or output from, processor 202 in the course of executing the computer-executable instructions.

Memory 204 may store a blood lactate level monitoring program that—upon execution by processor 202—performs one or more of the methods provided herein. In particular, in various embodiments provided herein, the blood lactate level monitoring program—upon execution by processor 202—implements a trained machine learning model that predicts, in real-time or near real-time time, patient blood lactate levels based on input data.

Communication interface 206 is one or more data network interface, such as an IEEE 802.3 or IEEE 802.11 interface, for communication over a network.

Display interface 208 is a display (e.g., a screen) suitable for outputting information and data for display to a user. In some cases, display 208 may display a graphical user interface (GUI) associated with one or more software programs stored on memory 208.

User input interface 210 is an interface used for receiving user inputs (e.g., keyboard, stylus, pointing device, touch pad, etc.). In some cases, the display interface 208 can be a touch screen display, and accordingly may function as a user input interface 210.

I/O interface 212 may be an interface for coupling one or more external devices to computing device 102. For example, as shown in FIG. 1, one or more sensors and/or monitoring devices 104 may be coupled directly to the computing device 102 via the I/O interface 212.

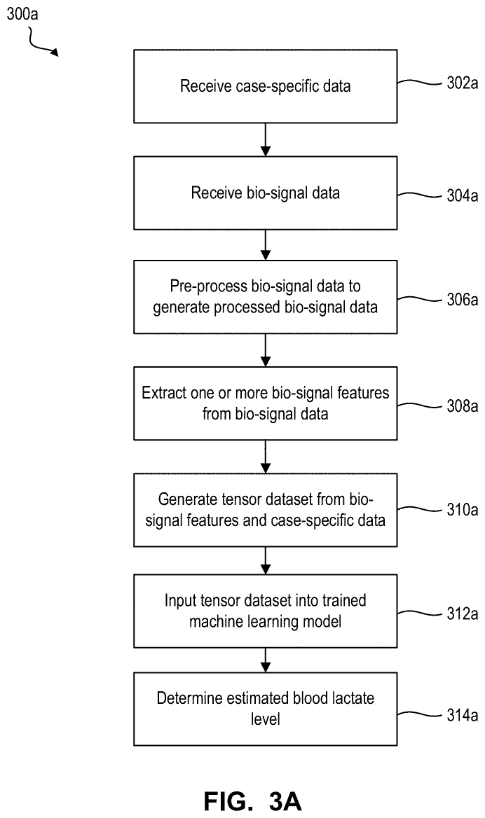

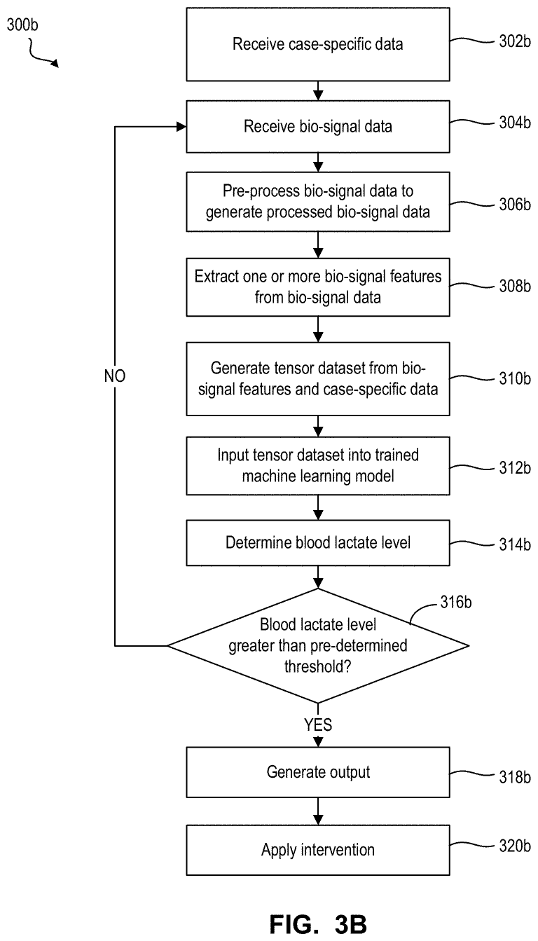

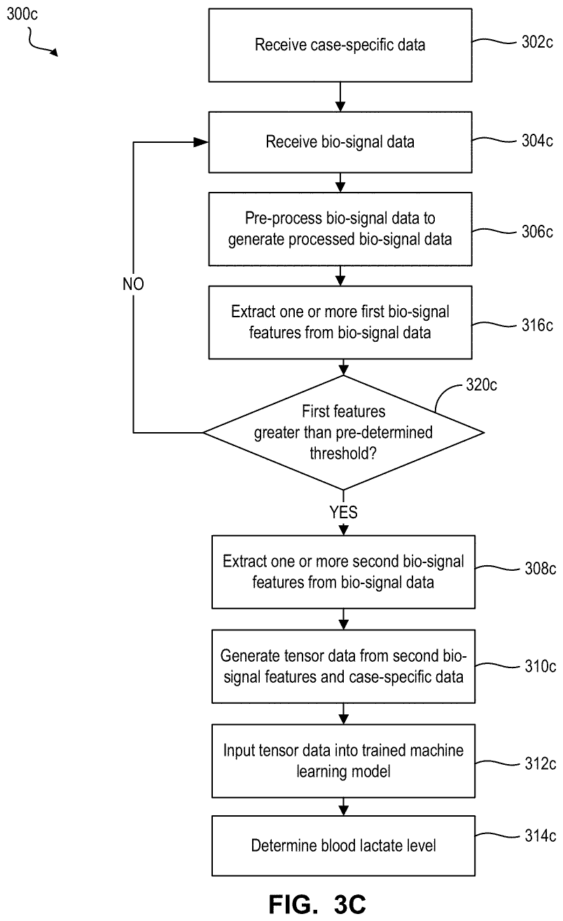

Referring now to FIG. 3A, there is shown an example process flow for a method 300a for automatic detection of blood lactate levels in a patient, according to at least some embodiments. Method 300 may be performed, for example, by executing—on the processor 202 of computing device 102—a blood lactate level monitoring program stored on computing device memory 204.

As shown, at 304, processor 202 may receive case-specific data associated with a patient whose blood lactate levels are being monitored (e.g., patient 106 in FIG. 1). In various cases, the case-specific data can include patient blood draw data as well as various other case-specific data as described herein.

Patient blood draw data can include, for example, one or more previously-determined blood lactate levels for the patient, based on physical test results, or as estimated using the techniques described herein. As provided in further detail herein, previously-determined patient blood lactate levels—and especially those based on physical test results—can be used as initial or anchoring values to allow a trained machine learning model to (continue to) predict or estimate the patient's current blood lactate levels at a given time.

The patient blood draw data can also include corresponding time indicia data. The time indicia data can include, for example, a timestamp indicating the acquisition time of the blood draw sample. Alternatively, or in addition, the time indicia can comprise an indication of the time elapsed since the previous blood draw sample(s) were acquired. As explained herein, the time indicia data may be used to increase the predictive accuracy of a trained machine learning model in estimating the patient's current blood lactate level using previously determined blood lactate levels.

Case-specific data received at 302a can also include various other biographical or observational data. For example, the case-specific data can include, by way of non-limiting examples, the patient's age, gender, weight, known disease status, hours since the patient was admitted into a clinical setting, various anatomical measurements, as well as various physiological or metabolic measurements of the patient.

In some embodiments, at 302a, some or all of the case-specific data may be received via the user input interface 210 of computing device 102. For example, the blood lactate level monitoring program—executing on processor 202—may display a graphical user interface (GUI) (e.g., on display interface 208), which may prompt a user (e.g., a clinician) to enter some or all of the case-specific data. Once entered, the case-specific data may be stored on device memory 204 for subsequent retrieval when needed, in which case it need not be re-entered.

The case-specific data may also be received from one or more external sources. For example, the case-specific data may be stored on, and retrieved from, database storage 110. Accordingly, the blood lactate level monitoring program—executing on processor 202—can transmit a request, e.g., via communication interface 206, to data storage 110, requesting the relevant case-specific data.

At 304a, bio-signal data may also be received by processor 202. The received bio-signal data can include—for example—patient ECG data (e.g., a PQRST waveform). For instance, the ECG data may be captured using one or more electrodes 104 applied to the patient's body 106 in known fashion. In other cases, the bio-signal data can include arterial pulse waveforms indicative of a patient's blood pressure. The arterial pulse waveform may be captured, for example, using a sphygmomanometer applied to the patient. In still other cases, the bio-signal data can include photoplethysmogram (PPG) captured by, for instance, a pulse oximeter. The bio-signal data may be received as a data array (e.g., numerical data array reflecting waveform amplitudes in time series form).

In some embodiments, the bio-signal data may be received at 304a in real-time, or near real-time, from one or more sensors and/or monitoring devices 104 applied to the patient. In other embodiments, the bio-signal data may have been previously captured, and stored on either the device memory 204, or database storage 110. In some cases, the bio-signal data may be stored, e.g., in the device memory 204 or database storage 110, in association with a corresponding acquisition time. Accordingly, at 304a, the stored bio-signal data could be retrieved in association with the acquisition time from the relevant memory or storage component.

In some embodiment, at 306a, the bio-signal data may undergo initial pre-processing. The pre-processing can include, for example, applying one or more filters to the bio-signal data. For instance, high-pass, low-pass, band-pass or other noise reducing filters can be applied to enhance signal quality or otherwise improve the signal to noise (SNR) ratio in the received data.

At 308a, the bio-signal data (i.e., pre-processed, or unprocessed bio-signal data) may be analyzed to extract one or more bio-signal data features.

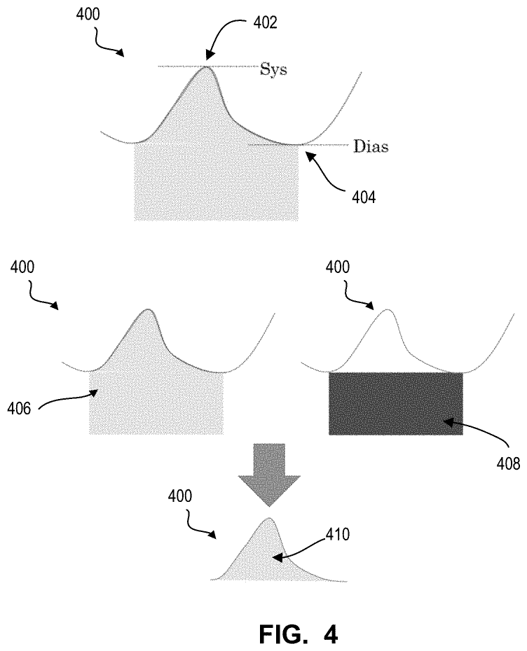

By way of example, where the bio-signal data comprises an arterial pulse waveform, the extracted features at 308a can include, for instance, patient heartbeat levels (e.g., based on the cardiac cycle frequency), as well as, for each cardiac cycle in the waveform, extracting (i) a systolic arterial pressure (P_{systolic}) value (402 in FIG. 4); (ii) a diastolic arterial pressure (P_{diastolic}) value (404 in FIG. 4); (iii) a mean arterial pressure (Pₘₑₐₙ) value; (iv) an area under the curve value (410 in FIG. 4); (v) a normalized area value; (vi) an area for one minute duration value; and (vii) a curve peak sharpness value.

Referring now briefly to FIG. 4, which diagrammatically illustrates an example arterial pulse waveform 400 representing example bio-signal data received at 308a.

As shown, in a given cardiac cycle, the pulse waveform 400 may oscillate between the systolic arterial pressure (P_{systolic}) point 402, and the diastolic arterial pressure (P_{diastolic}) point 404. The mean arterial pressure (Pₘₑₐₙ) of the cardiac cycle can be determined according to Equation (1).

$${P}_{m e a n} = \frac{{P}_{systoli c} + 2 ( {P}_{d iastoli c} )}{3} \qquad ( 1 )$$

The area 410 under the cardiac waveform curve 400 may be determined by subtracting the area 408 under the diastolic pressure (P_{diastolic}) point 404, from the area 406 under the systolic pressure (P_{systolic}) point 402. In cases where the cardiac waveform is sampled, the area under the curve—for a given cardiac cycle of the waveform—can also be determined according to Equation (2).

$$Curve Area = {\sum }_{n = 0}^{N} ( B P ( n ) - {P}_{d iastoli c} ) * Δ t \qquad ( 2 )$$

wherein N is the total number of samples in the sampled cardiac waveform; n is the n^{th} sample from 0 to N; BP(n) is the measured blood pressure at the n^{th} sample; P_{diastolic} is the measured diastolic blood pressure for a given cycle; and Δt is the sampling interval.

The normalized area under the curve may then be determined according to Equation (3):

$$A r e {a}_{normal i z e d} = \frac{Curve Area}{{P}_{systoli c} - {P}_{d iastoli c}} \qquad ( 3 )$$

In various cases, an area₍ₘᵢₙᵤₜₑ₎ value can also be determined, corresponding to an expanded value of the area for a one-minute interval of the cardiac waveform. The area₍ₘᵢₙᵤₜₑ₎ value can be determined in accordance with Equation (4):

$$A r e {a}_{minute} = Curve Area * \frac{60 , 000 ms}{Stroke Time Per Beat ( ms )} \qquad ( 4 )$$

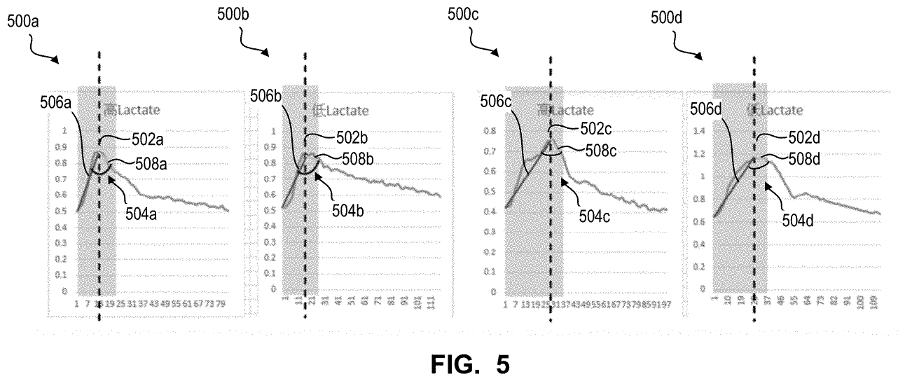

Referring now briefly to FIG. 5, which diagrammatically illustrates different arterial pulse waveforms 500a-500d having different waveform peak sharpness. In particular, FIG. 5 illustrates that the peak sharpness 502a-502d, for a cycle of the arterial waveform, can be determined as the angle 504a-504d between an upstroke line 506a-506d and a down stroke line 508a-508d of each waveform cycle.

Referring now back to FIG. 3A, at 308a, the one or more extracted data feature may also include data features extracted from other received bio-signal data (e.g., ECG or PPG waveforms), including extracting the waveform frequency, as well as for each cardiac cycle—extracting the maximum amplitude, minimum amplitude, mean amplitude, area under the curve, etc.

At 310a, a tensor dataset may be generated based on the case-specific data and the extracted bio-signal features. In general, a tensor dataset may refer to an N-dimensional matrix of data that aggregates each of the case-specific data and the extracted bio-signal features.

At 312a, the tensor dataset is input into a trained machine learning model. The trained model, in turn, is configured to process the case-specific data, as well as the extracted data features, to generate an estimated patient blood lactate level.

At 314a, a blood lactate level concentration is determined (e.g., estimated or predicted), based on the output of the trained machine learning model.

Accordingly, the method 300a may be used to predict or estimate a patient's current blood lactate levels based on new received bio-signal data, as well as previous patient blood lactate levels as determined from prior patient blood draw samples, as well as other case-specific data that may have also been received.

The blood lactate level determination at 314a may be displayed to a user (e.g., a medical practitioner) on a display interface 208 of the computing device 102. The blood lactate level also may be stored in memory 204 for subsequent retrieval. For example, the output blood lactate level may be stored in memory 204 in association with time indicia (e.g., a timestamp), and can be used as an input into the trained model in subsequent determinations of blood lactate levels based on new bio-signal data. In still other cases, the blood lactate level determination may be transmitted for storage on database storage 110.

In some embodiments, feature extraction at 308a may not be required, and the entire bio-signal data (e.g., a numerical array expressing one or more received waveforms) may be directly fed into the trained machine learning model at 310a. The machine learning model may then be configured to operate on unprocessed bio-signal data to generate an estimated blood lactate level for the patient. In these cases, the tensor dataset may not necessarily include extracted bio-signal features. In other cases, the bio-signal data, and one or more extracted features, can both be included in the input tensor dataset.

Referring now to FIG. 3B, there is shown an example process flow for a method 300b for automatic detection of blood lactate levels, according to some other embodiments. Method 300b may be performed, for example, by executing—on the computing device processor 202—the blood lactate level monitoring program stored on computing device memory 204.

Method 300b is generally analogous to method 300a, except that method 300b allows for generating an output when the blood lactate levels are determined to be above a predetermined threshold.

In particular, as shown, at 316b, it can be determined if the blood lactate level determined at 314b is above a predetermined threshold. This may indicate, for example, that the patient blood lactate levels are unsafe, and in particular, that the patient is in a pre-critical or critical state requiring intervention. For example, this can correspond to determining a blood lactate level greater than or substantially equal to 30 to 40 mg/dL (milligram per deciliter).

In cases where the determination at 316b is negative (e.g., blood lactate levels are below the predetermined threshold), the method 300b can return to 302b or 304b to receive new case-specific data or new bio-signal data, for generating subsequent blood lactate level estimates.

In cases where the determination at 316b is positive (e.g., blood lactate levels are above the predetermined threshold), an output can be generated at 318b. The output can correspond, for example, to a visual and/or audio notification to a user of computing device 102. For example, a visual notification may be displayed on display interface 208 and/or an audio notification may be generated using an audio speaker of computing device 102 (not shown).

In some cases, the output can be determined based on the specific patient blood lactate level. For example, lactate levels substantially equal to or greater than 40 mg/dL may indicate a critical patient state, and lactate levels substantially equal to or greater than 35 mg/dL and below 40 mg/dL can indicate a pre-critical patient state. Accordingly, the output can indicate the patient state (e.g., critical or pre-critical) based on the determined blood lactate level.

At 320b, responsive to the output, an intervention can be applied. The intervention can include, for example, providing an ameliorative drug to the patient to reduce blood lactate levels, or otherwise increasing oxygen supply to the patient. In some cases, the response can be based on the particular output (e.g., critical or pre-critical patient state). For example, a pre-critical state may result in preventive response measures (e.g., ameliorative drugs), while the critical state may result in more invasive response measures (e.g., surgery).

Referring now to FIG. 3C, there is shown a process flow for an example method 300c for automatic detection of patient blood lactate levels, according to some other embodiments. Method 300c may be performed as well, for example, by executing—on the processor 202—the blood lactate level monitoring program stored on computing device memory 204.

Method 300c is generally analogous to method 300a, except that at 316c, one or more first bio-signal features are extracted from the bio-signal data. In particular, specific features of bio-signal data may be important indicators of whether a patient's blood lactate level is within a critical range. For example, in various cases, sharp peaks in an arterial pulse waveform (e.g., 502 in FIG. 5) may be associated with an undesirable increase in the patient's blood lactate levels. Accordingly, specific bio-signal features can be monitored to trigger the analysis and determination of the patient's blood lactate level.

In particular, as shown, at 316c, it can be determined if the first extracted bio-signal features are above a predetermined threshold. For example, this can include determining a predetermined threshold for the peak sharpness or area under the curve in an arterial pulse waveform (e.g., a peaking sharpness of less than 30°-45°).

If this is not the case, the method 300c may return to monitoring patient bio-signal data at 304c (or receiving case-specific data at 302c). Otherwise, the method 300c may continue to acts 308c-314c, which are generally analogous to acts 308a-314a of method 300a. In particular, the method may involve analyzing one or more second bio-signal features at 308c, which can then be fed into the machine learning model to estimate blood lactate levels. In some cases, the first and second bio-signal features (316c and 308c) may overlap, wherein the first bio-signal features may represent a subset of the second bio-signal features. In some cases, method 300c may also include generating an output notification as shown in acts 316b and 318b of method 300b.

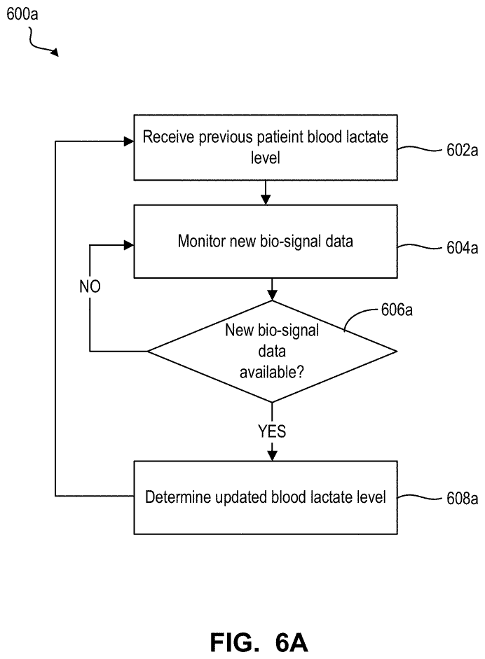

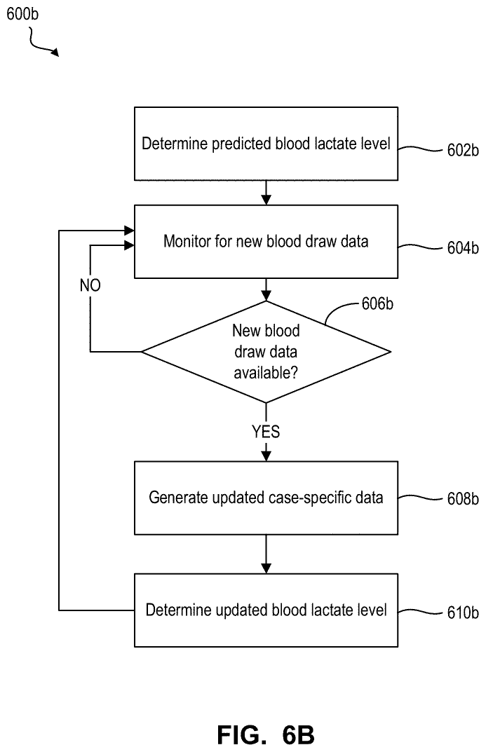

Referring now to FIG. 6A, there is shown a method 600a for real-time, or near real-time, monitoring of patient blood lactate levels based on monitored bio-signal data. Method 600a may be performed by executing the blood lactate level monitoring program on computer processor 202.

At 602a, a patient's previous blood lactate level is received. For example, this can correspond to a blood lactate level determined from previous patient blood draw data. In other cases, the blood lactate level received at 602a may correspond to a previous blood lactate level determined based on any one of methods 300a, 300b and/or 300c.

At 604a, the patient's bio-signal data is monitored, in real-time or near real-time, to determine if new (i.e., updated) patient bio-signal data is available. For example, as shown in FIG. 1, this can include continuous monitoring, in real-time or near real-time, bio-signal data outputs of one or more sensors or monitoring devices 104 applied to the patient 106.

At 606a, it is determined whether new bio-signal data is available (e.g., from sensors or monitoring devices 104). In some cases, the determination at 606a may involve a determination in respect of any new bio-signal data. In other cases, 606a may require a determination in respect of at least one new available cardiac cycle. For example, at least one cardiac cycle may be required to allow extraction of relevant features from the bio-signal data as previously provided in methods 300a-300c.

At 608a, an updated blood lactate level estimate is determined based on the new bio-signal data, in accordance with any one of methods 300a-300c. Once an updated blood lactate level is determined at 608a, the method 600a may return to 602a where the updated blood lactate level may now correspond to a previous determined blood lactate level in the next iteration of method 600a. In other cases, the method 600a may simply return to monitoring for new bio-signal data.

In view of the foregoing, the method 600a allows real-time or near real-time implementations of the methods 300a-300c for determining patient blood lactate level based on monitored patient bio-signal data.

Referring now to FIG. 6B, there is shown a method 600b for updating blood lactate prediction estimates based on new patient blood draw data. Method 600b may be performed, for example, by executing—on the computing device processor 202—the blood lactate level monitoring program stored on computing device memory 204.

In particular, method 600b can be used to occasionally correct (e.g., refine) estimates generated by the trained machine learning models in methods 300a-300c based on new available patient blood draw data. Over time, the estimated blood lactate level—generated by a trained machine learning model—may deviate from the patient's true blood lactate level. Accordingly, blood lactate levels, determined from new patient blood draw samples, can be inputs into the trained model to correct the model, and thus enhance the model's predictive accuracy over time.

As shown, at 602b, an estimated blood lactate level concentration can be determined using any one of methods 300a-300c.

At 604b, the blood lactate level detection application can monitor for new blood draw data associated with the patient. For example, the monitoring can involve monitoring for new user inputs (e.g., via user input interface 210) in respect of new blood draw data. In other cases, the monitoring can involve requesting new blood draw data stored on the database storage 110.

At 606b, based on the monitoring, it can be determined whether new blood draw data is available. If no new blood draw data is available, the method 600b can return to repeat from act 604b. In some cases, the iteration between 604b and 606b may occur continuously, or at pre-defined time or frequency intervals.

At 608b, if new blood draw data is available, updated case-specific data can be generated, which includes the new blood draw data (e.g., new blood lactate level determinations, and corresponding time indicia).

At 610b, any one of methods 300a-300c can be performed using the updated case-specific data to generate an updated estimated blood lactate level determination.

Acts 604b and 606b of methods 600b can also be performed in respect of any other new case-specific data (e.g., updated patient weight, age, medical status, etc.) to ensure that the trained model is receiving accurate and up-to-date case-specific data.

Methods 600a and 600b may occur concurrently, such that updated blood lactate levels can be determined based on both new patient bio-signal data (FIG. 6A), as well as updated case-specific data (FIG. 6B).

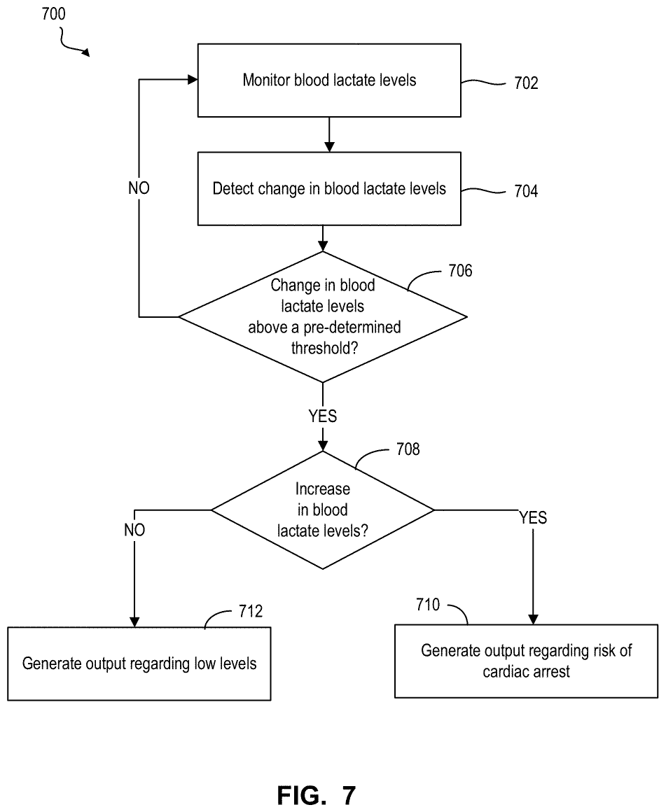

Referring now to FIG. 7, there is shown a method 700 for predicting the risk of cardiac arrest based on detected blood lactate levels, according to some embodiments. In at least some embodiments, the method 700 can be performed in real-time, or near real-time. Method 700 can be implemented, for example, using the processor 202 of computing device 102.

At 702, patient blood lactate levels can be monitored using, for example, any one of the methods 300a-300c, 600a and/or 600b.

At 704, based on the monitoring, it can be determined if there is a detected change to the estimated patient blood lactate levels from a previously determined blood lactate level.

At 707, it can be determined if the change in blood lactate levels is above a predetermined threshold (e.g., substantially equal or greater than 35-40 mg/dL). If the change is not above a predetermined threshold, the method 700 can return to 702 to iterate. In various cases, the method may iterate continuously, or otherwise at pre-defined frequency or time intervals.

Otherwise, at 706, it can be determined if the change corresponds to an increase in blood lactate levels. At 708, if the change corresponds to an increase, an output (e.g., audio and/or visual notification) can be generated to alert a user of the computing device 102 in regards of the patient being at risk of cardiac arrest. Otherwise, at 710, if the changes do not correspond to an increase (e.g., a decrease or no change), then an output can be generated regarding low lactate levels (e.g., indicating low risk of patient problems, i.e., cardiac arrest).

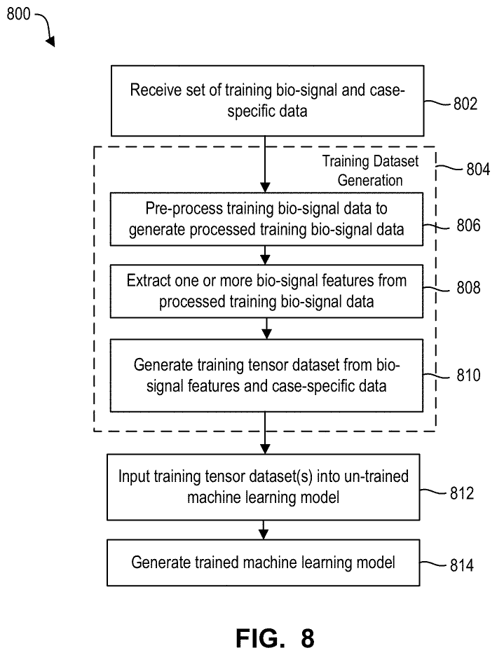

Referring now to FIG. 8, there is shown an example process flow for a method 800 for training a machine learning model for automatic detection of patient blood lactate levels, for use with the embodiments described herein. As described, the trained machine learning model can also be used to predict patient risk of cardiac arrest, based on changes in predicted blood lactate levels.

Method 800 may be performed on the processor 202 of the computing device 102. In other cases, the method 800 may be performed on an external server (e.g., associated with database storage 110). In the latter case, once the trained model is generated, its parameters may be transmitted to the computing device 102 for storage on memory 204 and for use in predicting patient blood lactate levels in accordance with the methods provided herein.

As shown, at 802, a training data set is received. The training data set comprises case-specific data and bio-signal data as described herein. In some cases, the bio-signal training data may be based on previously acquired data from patients whose blood lactate level has been previously determined.

At 804, based on the training data, a training dataset is generated for use in training a machine learning model. The process of generating the training dataset can include—for each training set—at 806, pre-processing the bio-signal data to generate pre-processed bio-signal data (e.g., applying one or more noise filters). In some embodiments, the pre-processing at 806 may be omitted.

At 808, the pre-processed training bio-signal data may be filtered to extract one or more bio-signal data features in a manner analogous to act 308a of method 300a. In some cases, feature extraction at 808 may also be omitted where the machine learning model is trained using the complete bio-signal dataset.

At 810, training tensor datasets are generated based on the extracted bio-signal features (and/or complete bio-signal data), and the case-specific training data. In various cases, a plurality of training tensor datasets are generated in association with each patient profile received at 802.

At 812, the training tensor datasets may be input into an un-trained machine learning model. Using one of a variety of suitable machine learning techniques, the model may be trained to correlate various case-specific data, as well as bio-signal data and/or extracted bio-signal data features to estimated blood lactate level concentrations.

Various types of supervised machine learning algorithms can be used at 812 to train the machine learning model, including models based on Naïve-Bayes, K-Nearest Neighbor (KNN) method, convolution neural network (CNN) methods, support vector machines (SVM), boosting and adaptive boosting techniques, cross-validation techniques (e.g., K-fold with K=1-20, Efron's bootstrap, etc.), recurrent neural networks (RNNs) and random forest methods. In at least some embodiments, the machine learning model can include a random forest technique that is hypertuned (i.e., the model parameters of the model are set by the training algorithim). In some embodiments, where the machine learning model is receiving unprocessed bio-signal data, the machine learning model can comprise a deep learning model (e.g., a CNN) which is configured to learn to extract relevant bio-signal data features for predicting blood lactate levels.

At 814, based on the training, a trained machine learning algorithm may be generated for estimating patient blood lactate levels.

It will be appreciated that numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail since these are known to those skilled in the art. Furthermore, it should be noted that this description is not intended to limit the scope of the embodiments described herein, but rather as describing exemplary implementations. Various modification and variations may be made to these example embodiments without departing from the spirit and scope of the invention, which is limited only by the appended claims.

1. A method of determining blood lactate levels, the method comprising:
receiving bio-signal data associated with a patient;
processing the bio-signal data to extract one or more bio-signal data features;
generating a tensor dataset comprising the one or more bio-signal data features; and
processing the tensor dataset using a machine learning model to generate an estimated blood lactate level.

2. The method of claim 1, wherein the method further comprises:
based on the estimated blood locate level, generating a prediction of risk of cardiac arrest for the patient.

3. The method of claim 1, where the patient is a pediatric patient.

4. The method of claim 1, further comprising updating the tensor dataset to comprise case-specific data, prior to processing the tensor dataset.

5. The method of claim 4, wherein the case-specific data comprises previous blood draw data for the patient.

6. The method of claim 4, wherein the case-specific data comprises at least one of biographical data and observational data for the patient.

7. The method of claim 1, wherein the machine learning model is a convolutional neural network.

8. The method of claim 1, wherein the machine learning model is based on support vector machines.

9. The method of claim 1, wherein the machine learning model is generated using a random forest algorithm.

10. The method of claim 1, wherein the processing of the tensor dataset comprises using cross-validation.

11. The method of claim 10, wherein the cross-validation comprises one of K-fold and Efron's bootstrap.

12. The method of claim 11, wherein the cross-validation is K-fold cross-validation, and K is in a range of 1 to 20.

13. The method of claim 1, wherein the machine learning model is determined using adaptive boosting.

14. The method of claim 1, wherein the one or more bio-signal data features comprise second bio-signal data features, and the method further comprises:
processing the bio-signal data to generate one or more first bio-signal data features;
determining if the one or more first bio-signal data features are greater than a predetermined threshold; and
in response to the determination, processing the tensor dataset using the machine learning model.

15. The method of claim 1, further comprising applying a treatment to the pediatric patient in response to the estimated blood lactate level.

16. The method of claim 1, wherein the bio-signal data comprises at least one arterial blood pressure data or electrocardiogram (ECG) data.

17. A non-transitory computer readable medium storing computer program instructions which, when executed by at least one processor, cause the at least one processor to carry out the method comprising:
receiving bio-signal data associated with a patient;
processing the bio-signal data to extract one or more bio-signal data features;
generating a tensor dataset comprising the one or more bio-signal data features; and
processing the tensor dataset using a machine learning model to generate an estimated blood lactate level.

18. A system for determining blood lactate levels, the system comprising:
at least one processor; and
a memory coupled to the at least one processor;
the at least one processor being configured to carry out the method comprising:
receiving bio-signal data associated with a patient;
processing the bio-signal data to extract one or more bio-signal data features;
generating a tensor dataset comprising the one or more bio-signal data features; and
processing the tensor dataset using a machine learning model to generate an estimated blood lactate level.
